SEQUENCE LISTING (10 sequences)

SEQ ID NO: 1 - RNA, 16 nt, Homo sapiens
  source 1..16: mol_type = transcribed RNA
cagatcacagctctta

SEQ ID NO: 2 - RNA, 17 nt, Homo sapiens
  source 1..17: mol_type = transcribed RNA
agatcacagctcttaaa

SEQ ID NO: 3 - RNA, 18 nt, Homo sapiens
  source 1..18: mol_type = transcribed RNA
atcacagctcttaaaatt

SEQ ID NO: 4 - RNA, 17 nt, Homo sapiens
  source 1..17: mol_type = transcribed RNA
ttttcagatcacagctc

SEQ ID NO: 5 - RNA, 16 nt, Homo sapiens
  source 1..16: mol_type = transcribed RNA
tttcagatcacagctc

SEQ ID NO: 6 - RNA, 17 nt, Homo sapiens
  source 1..17: mol_type = transcribed RNA
tttcagatcacagctct

SEQ ID NO: 7 - RNA, 15 nt, Homo sapiens
  source 1..15: mol_type = transcribed RNA
ttcagatcacagctc

SEQ ID NO: 8 - RNA, 16 nt, Homo sapiens
  source 1..16: mol_type = transcribed RNA
ttcagatcacagctct

SEQ ID NO: 9 - RNA, 2472 nt, Homo sapiens
  source 1..2472: mol_type = transcribed RNA
agaaggggttgttttgcttttgcttatttccgtccatttccctctctgcgcgcggaccttccttttccagatggtgagagccgcggggacacccgacgccggggcaggctgatccacgatcctgggtgtgcgtaacgccgcctggggctccgtgggcgagggacgtgtggggacaggtgcaccggaaactgccagactggagagttgaggcatcggaggcgcgagaacagcactactactgcggcgagacgagcgcggcgcatcccaaagcccggccaaatgcgctcgtccctgggaggggagggaggcgcgcctggagcggggacagtcttggtccgcgccctcctcccgggtctgtgccgggacccgggacccgggagccgtcgcaggtctcggtccaaggggccccttttctcggaagggcggcggccaagagcagggaaggtggatctcaggtagcgagtctgggcttcggggacggcggggaggggagccggacgggaggatgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgcttatcagaacgaggagatcacagctcttaaaattaagtacaatccatttgcaaaagctttccttgatgcaaaggaaagaagtgatcacaaagagatgatggaggaacccggagacagccagcaacctgggtactcccaatgggggtggcttcttcctggaaccagcaccctgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtgaagcagcaaggcccaggtcccgaaagatgcagtgactttttgtcgtggcagccagtggtgactggattgacctactaggtacccagtggcagtctcaggttaagaaggaaatgcagcctcagtaacttccttttcaaagcagtggaggagcacacggcacctttccccagagccccagcatcccttgctcacacctgcagtagcggtgctgtcccaggtggcttacagatgaacccaactgtggagatgatgcagttggcccaacctcactgacggtgaaaaaatgtttgccagggtccagaaactttttttggtttatttctcatacagtgtattggcaactttggcacaccagaatttgtaaactccaccagtcctactttagtgagataaaaagcacactcttaatcttcttccttgttgctttcaagtagttagagttgagctgttaaggacagaataaaatcatagttgaggacagcaggttttagttgaattgaaaatttgactgctctgccccctagaatgtgtgtattttaagcatatgtagctaatctcttgtgttgttaaactataactgtttcatatttttcttttgacaaagtagccaaagacaatcagcagaaagcattttctgcaaaataaacgcaatatgcaaaa

SEQ ID NO: 10 - protein, 435 aa, Homo sapiens
MSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGGPQRMITSHCFPETQFIAVTAYQNEEITALKIKYNPFAKAFLDAKERSDHKEMMEEPGDSQQPGYSQWGWLLPGTSTLCPPANPHPQFGGALSLPSTHSCDRYPTLRSHRSSPYPSPYAHRNNSPTYSDNSPACLSMLQSHDNWSSLGMPAHPSMLPVSHNASPPTSSSQYPSLWSVSNGAVTPGSQAAAVSNGLGAQFFRGSPAHYTPLTHPVSAPSSSGSPLYEGAAAATDIVDSQYDAAAQGRLIASWTPVSPPSM

(19) United States
(12) Patent Application Publication
(10) Pub. No.: US 2026/0201385 A1
(43) Pub. Date: Jul. 16, 2026

(54) Inhibitory Nucleic Acids and Methods of Use Thereof

(71) Applicant: The Regents of the University of California, Oakland, CA (US)

(72) Inventors: Anders Michael Näär, Berkeley, CA (US); Caslin Anne Gilroy, Oakland, CA (US)

(21) Appl. No.: 19/135,691

(22) PCT Filed: Dec. 5, 2023
(86) PCT No.: PCT/US2023/082556

§ 371 Date: Jun. 4, 2025

Related U.S. Application Data
(60) Provisional application No. 63/430,731, filed on Dec. 7, 2022.

(51) Int. Cl. C12N 15/113; A61K 9/51; A61K 31/712; A61K 31/7125; A61K 45/06; A61P 35/00

(52) U.S. Cl. CPC C12N 15/1136; A61K 9/5123; A61K 31/712; A61K 31/7125; A61K 45/06; A61P 35/00; C12N 2310/11; C12N 2310/14; C12N 2310/315; C12N 2310/3231; C12N 2320/33

(57) ABSTRACT

The present disclosure provides inhibitory nucleic acids, compositions comprising the inhibitory nucleic acids, and methods of using the inhibitory nucleic acids to treat carcinomas.

### CROSS-REFERENCE

This application claims the benefit of U.S. Provisional Patent Application No. 63/430,731, filed Dec. 7, 2022, which application is incorporated herein by reference in its entirety.

### INCORPORATION-BY-REFERENCE OF MATERIAL ELECTRONICALLY SUBMITTED

A Sequence Listing is provided herewith as a Sequence Listing XML, “BERK-480WO_SEQ_LIST” created on Dec. 1, 2023 and having a size of 12,658 bytes. The contents of the Sequence Listing XML are incorporated by reference herein in their entirety.

### INTRODUCTION

Brachyury (also known as “TBXT”) has been shown to be overexpressed in carcinomas of the lung, breast, colon, prostate, and liver, as well as in chordoma. Brachyury has been associated with the epithelial-mesenchymal transition in human tumors, and is under investigation as a drug target for a wide range of carcinomas.

Chordoma is a malignant sarcoma originating from notochord cells in the vertebrae and is rare (1:1,000,000), with 300 diagnoses per year in the United States. A chordoma tumor is slow growing; however, treatment is challenging due to the proximity of the tumor to the spinal cord, and the tendency for recurrence following surgery and radiation. There are no systemic targeted therapies for chordoma, and consequently, patients with advanced and metastatic disease face poor prognoses.

There is a need in the art for compositions and methods for treating carcinomas.

### SUMMARY

The present disclosure provides inhibitory nucleic acids, compositions comprising the inhibitory nucleic acids, and methods of using the inhibitory nucleic acids to treat carcinomas.

### BRIEF DESCRIPTION OF THE DRAWINGS

FIG. 1 is a schematic depiction of TBXT-targeting mechanism of an antisense oligonucleotide (ASO) library.

FIG. 2 depicts the effect of select mixmer locked nucleic acid (LNA) ASOs on TBXT expression in UM-Chor1 cells.

FIG. 3 depicts localization of anti-TBXT LNA ASO mixmers to the same TBXT exon.

FIG. 4 depicts non-specific toxicity of anti-TBXT LNA ASO mixmer hits in U2OS cells.

FIG. 5 depicts inhibition of TBXT expression by select mixmer LNA ASOs in UM-Chor1 cells.

FIG. 6 depicts non-specific toxicity of anti-TBXT LNA ASO mixmer redesigns in U2OS cells.

FIG. 7 depicts inhibition of TBXT expression be nontoxic mixmer LNA ASO redesigns in UM-Chor1 cells.

FIG. 8 depicts inhibition of TBXT expression in MUG-Chor1 cells by original and redesign LNA ASOs.

FIG. 9 depicts inhibition of TBXT expression in JHC7 cells by original and redesign LNA ASOs.

FIG. 10 depicts inhibition of TBXT expression in UCH-2 cells by original and redesign LNA ASOs.

FIG. 11 provides an anti-TBXT mixmer LNA ASO screen overview.

FIG. 12A-12B depict inhibition of UM-Chor1 chordoma cell growth by anti-TBXT mixmer LNA ASOs.

FIG. 13 provides a TBXT nucleotide sequence (SEQ ID NO:9).

FIG. 14 provides a TBXT amino acid sequence (SEQ ID NO:10).

FIG. 15A-15B depicts entry of an anti-TBXT mixmer LNA ASO into chordoma cells without a lipid delivery vehicle.

FIG. 16A-16D depicts exon 5 skipping in TBXT transcripts induced by anti-TBXT mixmer LNA ASOs.

FIG. 17A-17B depicts induction of S-phase cell cycle arrest in chordoma cells by an anti-TBXT mixmer LNA ASO.

FIG. 18A-18E depicts tolerance of an anti-TBXT mixmer LNA ASO in mice.

FIG. 19A-19D depicts eradication of patient-derived chordoma tumors in mice by an anti-TBXT mixmer LNA ASO.

### DEFINITIONS

As used herein, an “antisense oligonucleotide” (“ASO”) refers to a nucleic acid sequence that is complementary to a DNA or RNA sequence.

“RNA” refers to a molecule comprising at least one or more ribonucleotide residues. A “ribonucleotide” is a nucleotide with a hydroxyl group at the 2′ position of a beta-D-ribofuranose moiety. The term RNA, as used herein, includes double-stranded RNA, single-stranded RNA, isolated RNA, such as partially purified RNA, essentially pure RNA, synthetic RNA, recombinantly produced RNA, as well as altered RNA that differs from naturally occurring RNA by the addition, deletion, substitution and/or alteration of one or more nucleotides. Nucleotides of the RNA molecules can also comprise non-standard nucleotides, such as non-naturally occurring nucleotides or chemically synthesized nucleotides or deoxynucleotides.

A “microRNA” (miRNA) is a single-stranded RNA molecule of about 21-23 nts in length. In general, miRNAs regulate gene expression. miRNAs are encoded by genes from whose DNA they are transcribed, but miRNAs are not translated into protein. Each primary miRNA transcript is processed into a short stem-loop structure before undergoing further processing into a functional miRNA. Mature miRNA molecules are partially complementary to one or more messenger RNA (mRNA) molecules, and their main function is to down-regulate gene expression.

As used herein “an interfering RNA” refers to any double stranded or single stranded RNA sequence, capable, either directly or indirectly (i.e., upon conversion), of inhibiting or down regulating gene expression by mediating RNA interference. Interfering RNA includes but is not limited toenail interfering RNA (“siRNA”) and small hairpin RNA (“shRNA”). “RNA interference” refers to the selective degradation of a sequence-compatible messenger RNA transcript.

As used herein “an shRNA” (small hairpin RNA) refers to an RNA molecule comprising an antisense region, a loop portion and a sense region, wherein the sense, region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family.

A “small interfering RNA” or “siRNA” as used herein refers to any small RNA molecule capable of inhibiting or down regulating gene expression by mediating RNA interference in a sequence specific manner. The small RNA can be for example, about 18 to 21 nucleotides long.

As used herein, an “antagomir” refers to a small synthetic RNA having complementarity to a specific microRNA target, with either mispairing at the cleavage site or one or more base modifications to inhibit cleavage.

As used herein, the phrase “post-transcriptional processing” refers to mRNA processing that occurs after transcription and is mediated, for example, by the enzymes Dicer and/or Drosha.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to an individual organism, e.g., a mammal, including, but not limited to, murines, simians, humans, and non-human primates. In some cases, an “individual” is a human.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an antisense oligonucleotide (ASO)” includes a plurality of such ASOs and reference to “the TBXT polypeptide” includes reference to one or more TBXT polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that aspects and embodiments of the present disclosure described herein include “comprising,” “consisting,” and “consisting essentially of” aspects and embodiments.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides inhibitory nucleic acids, compositions comprising the inhibitory nucleic acids, and methods of using the inhibitory nucleic acids to treat carcinomas.

### Inhibitory Nucleic Acids

The present disclosure provides inhibitory nucleic acids that provide for a reduction in the level of a TBXT polypeptide in a cell.

TBXT is also referred to in the art as “T-Box Transcription Factor T,” “brachyury protein,” “SAVA,” and “FTFT.” A TBXT polypeptide can have at least 75%, at least 80%, at least 85%, at least 90%, at least 95%, at least 98%, at least 99%, or 100%, amino acid sequence identity to the TBXT amino acid sequence depicted in FIG. 14.

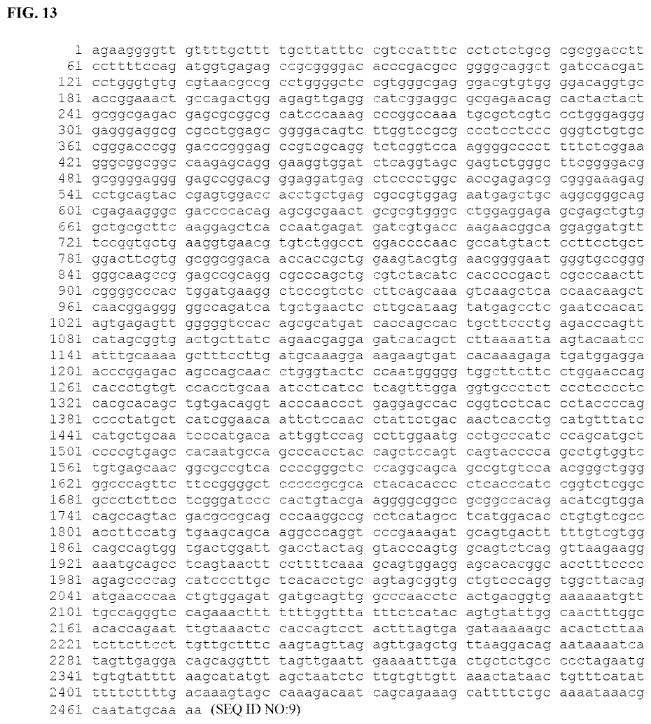

An inhibitory nucleic acid of the present disclosure comprises a nucleotide sequence that binds to (hybridizes with) a target TBXT nucleic acid. In some cases, the target TBXT nucleic acid is a TBXT polypeptide-encoding mRNA. In some cases, the target TBXT nucleic acid is a TBXT pre-mRNA. In some cases, the target TBXT nucleic acid comprises the intron 4/exon 5 junction of a TBXT mRNA. A TBXT genomic nucleotide sequence is provided in NCBI Gene ID 6862. A target TBXT nucleotide sequence can have at least 75%, at least 80%, at least 85%, at least 90%, at least 95%, at least 98%, at least 99%, or 100%, nucleotide sequence identity to the TBXT nucleotide sequence depicted in FIG. 13. In some cases, the target TBXT nucleic acid comprises the nucleotide sequence CAGATCACAGCTCTTA (SEQ ID NO:1). In some cases, the target TBXT nucleic acid comprises the nucleotide sequence AGATCACAGCTCTTAAA (SEQ ID NO:2). In some cases, the target TBXT nucleic acid comprises the nucleotide sequence ATCACAGCTCTTAAAATT (SEQ ID NO:3). In some cases, the target TBXT nucleic acid comprises the nucleotide sequence TTCAGATCACAGCTC (SEQ ID NO:7). In some cases, an inhibitory nucleic acid of the present disclosure binds to (hybridizes with) the TBXT target sequence CAGATCACAGCTCTTA (SEQ ID NO:1). In some cases, an inhibitory nucleic acid of the present disclosure binds to (hybridizes with) the TBXT target sequence AGATCACAGCTCTTAAA (SEQ ID NO:2). In some cases, an inhibitory nucleic acid of the present disclosure binds to (hybridizes with) the TBXT target sequence ATCACAGCTCTTAAAATT (SEQ ID NO:3). In some cases, an inhibitory nucleic acid of the present disclosure binds to (hybridizes with) the TBXT target sequence TTTTCAGATCACAGCTC (SEQ ID NO:4). In some cases, an inhibitory nucleic acid of the present disclosure binds to (hybridizes with) the TBXT target sequence TTTCAGATCACAGCTC (SEQ ID NO:5). In some cases, an inhibitory nucleic acid of the present disclosure binds to (hybridizes with) the TBXT target sequence TTTCAGATCACAGCTCT (SEQ ID NO:6). In some cases, an inhibitory nucleic acid of the present disclosure binds to (hybridizes with) the TBXT target sequence TTCAGATCACAGCTC (SEQ ID NO:7). In some cases, an inhibitory nucleic acid of the present disclosure binds to (hybridizes with) the TBXT target sequence TTCAGATCACAGCTCT (SEQ ID NO:8).

An inhibitory nucleic acid of the present disclosure reduces the level of a TBXT polypeptide in a cell (e.g., a target cell, such as a carcinoma, e.g., a chordoma) by at least 10%, at least 15%, at least 20%, at least 25%, at least 30%, at least 35%, at least 40%, at least 45%, at least 50%, at least 60%, at least 65%, at least 70%, or more than 70%, compared to the level of the TBXT polypeptide in the cell in the absence of the inhibitory nucleic acid. In some cases, an inhibitory nucleic acid of the present disclosure inhibits splicing of a TBXT transcript. In some cases, an inhibitory nucleic acid of the present disclosure blocks translation of a TBXT mRNA. In some cases, an inhibitory nucleic acid of the present disclosure increases cleavage of a TBXT mRNA by an RNAse H.

In some cases, an inhibitory nucleic acid of the present disclosure inhibits growth of a carcinoma by at least 10%, at least 15%, at least 20%, at least 25%, at least 30%, at least 35%, at least 40%, at least 45%, at least 50%, at least 60%, at least 65%, at least 70%, or more than 70%, compared the level of growth of the carcinoma in the absence of the inhibitory nucleic acid.

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), and other oligomeric compounds or oligonucleotide mimetics which hybridize to at least a portion of the target nucleic acid (i.e., a TBXT nucleic acid) and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010040112. In some cases, an inhibitory nucleic acid of the present disclosure is an ASO.

In some cases, the inhibitory nucleic acids are 10 to 50, 13 to 50, or 13 to 30 nucleotides in length. One having ordinary skill in the art will appreciate that this embodies oligonucleotides having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some cases, an inhibitory nucleic acid of the present disclosure is 15 nucleotides in length. In some cases, an inhibitory nucleic acid of the present disclosure is 12 to 30 or 13 to 30 nucleotides in length. One having ordinary skill in the art will appreciate that this embodies inhibitory nucleic acids having a length of 12, 13, 14, 15, 16, 17, 18, 19, 20, 21, 22, 23, 24, 25, 26, 27, 28, 29 or 30 nucleotides.

In some cases, the inhibitory nucleic acid of the present disclosure comprises a nucleotide sequence selected from:

+T*+A*A*G*+A*G*C*+T*G*T*+G*A*T*+C*+T*+G;
+T*+T*T*A*+A*G*A*+G*C*T*+G*T*G*+A*T*+C*+T;
+A*+A*T*T*+T*T*A*+A*G*A*+G*C*T*+G*T*+G*+A*+T;
+G*+A*+G*C*+T*G*T*+G*A*T*+C*+T*G*+A*A*+A*+A;
+G*+A*G*+C*+T*+G*T*+G*A*T*+C*T*G*+A*+A*+A;
+G*+A*G*C*+T*G*T*+G*A*+T*+C*T*+G*+A*+A*+A;
+A*+G*+A*G*C*+T*G*T*+G*A*T*+C*T*G*+A*+A*+A;
+A*+G*+A*G*C*+T*G*T*+G*A*T*+C*T*+G*A*+A*+A;
+G*+A*G*C*+T*G*+T*+G*A*T*+C*T*G*+A*+A;
and
+A*+G*+A*G*C*+T*G*T*+G*A*T*+C*T*G*+A*+A,

wherein * indicates a phosphorothioate linkage, and wherein “+” preceding a nucleotide indicates that the nucleotide is an LNA. In some cases, the inhibitory nucleic acid has a length of from 15 nucleotides to 25 nucleotides (e.g., 15, 16, 17, 18, 19, 20, 21, 22, 23, 24, or 25 nucleotides).

In some cases, the inhibitory nucleic acids are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the present disclosure may be formed as composite structures of two or more oligonucleotides, modified oligonucleotides, oligonucleosides and/or oligonucleotide mimetics as described above. Such compounds have also been referred to in the art as hybrids or gapmers.

In some embodiments, the inhibitory nucleic acid comprises at least one nucleotide modified at the 2 position of the sugar, e.g., a 2′-O-alkyl, 2′-O-alkyl-O-alkyl or 2′-fluoro-modified nucleotide. In other cases, RNA modifications include 2′-fluoro, 2′-amino and 2′O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than: 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. In some cases, inhibitory nucleic acids are oligonucleotides with phosphorothioate backbones and those with heteroatom backbones, particularly CH₂—NH—O—CH₃, CH₃—N(CH₃)—O—CH₂ (known as a methylene(methylimino) or MMI backbone], CH₂—O—N(CH₃)—CH2, CH₂—N(CH₃)—N(CH₃)—CH₂ and O—N(CH₃)—CH₂—CH₂ backbones, wherein the native-phosphodiester backbone is represented as O—P—O—CH); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters; aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide; sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

One or more substituted sugar moieties can also be included, e.g., one of the following at the 2′ position: OH, SH, SCH₃, F, OCN, OCH₃OCH₃, OCH₃, O(CH₂)n CH₃, O(CH₂)nNH₂ or O(CH₂)nCH₃ where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂ CH₃; ONO₂; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A suitable modification includes 2′-methoxyethoxy [2′-O—CH₂CH₂OCH₃, also known as: 2′-O-(2-methoxyethyl)](Martin et al, Helv. Chim. Acta, 1995, 78, 486). Other modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine, (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. A “universal” base known in the art, e.g., inosine, can also be included. 5-Me-C substitutions have been shown to increase nucleic acid duplex stability by 0.6-12<0>C. In some cases, an inhibitory nucleic acid of the present disclosure comprises one or more 5-Me-C.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by English et al., Angewandle Chemie, International Edition’, 1991, 30, page 613, and those disclosed by Sanghvi, Y. S., Chapter 15, Antisense Research and Applications', pages 289-302, Crooke, S. T. and Lebleu, B. ea., CRC Press, 1993. Certain of these nucleobases are particularly useful for increasing the binding affinity of an inhibitory nucleic acid. These include 5-substituted pyrimidines, 6-azapyrimidines and N-2, N-6 and 0-6 substituted purines, comprising 2-aminopropyladenine, 5-propynyluracil and 5-propynylcytosine, 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are suitable for inclusion in an inhibitory nucleic acid, e.g., alone or together with 2′-O-methoxyethyl sugar modifications.

In some cases, the inhibitory nucleic acids are chemically linked to one or more, moieties or conjugates that enhance the activity, cellular distribution, or cellular uptake of the oligonucleotide. Such moieties comprise but are not limited to, lipid moieties such as a cholesterol moiety; cholic acid; a thioether, e.g., hexyl-S-tritylthiol; a thiocholesterol; an aliphatic chain, e.g., dodecandiol or undecyl residues; a phospholipid; e.g., di-hexadecyl-rac-glycerol or triethylammonium 1,2-di-O-hexadecyl-rac-glycero-3-H-phosphonate; a polyamine or a polyethylene glycol chain; adamantane acetic acid; a palmityl moiety; an octadecylamine moiety; or a hexylamino-carbonyl-t oxycholesterol moiety.

These moieties or conjugates can include conjugate groups covalently bound to functional groups such as primary or secondary hydroxyl groups. Conjugate groups suitable for use include intercalators, importer molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation and/or strengthen sequence-specific hybridization with the target nucleic acid. Groups that enhance the pharmacokinetic properties include groups that improve uptake, distribution, metabolism or excretion of an inhibitory nucleic acid. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety.

The inhibitory nucleic acids useful in the present methods are sufficiently complementary to all or part of a TBXT nucleic acid, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base, al one position of an inhibitory nucleic acid is capable of hydrogen bonding with a base at the corresponding position of a TBXT sequence, then the bases are considered to be complementary to each other at that position. 100% complementary to is not required.

In the context of this disclosure, hybridization, means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. The inhibitory nucleic acids and the TBXT nucleic acid are complementary to each other when a sufficient number of corresponding positions in each molecule are occupied, by nucleotides that can hydrogen bond with each other. Thus, “specifically hybridizable” and “complementary” are terms which are used to indicate a sufficient degree of complementarity or precise pairing such that stable and specific binding occurs between the inhibitory nucleic acid and the TBXT target sequence. For example, if a base at one position of an inhibitory nucleic acid is capable of hydrogen bonding with a base at the corresponding position of a TBXT nucleic acid molecule; then the bases are considered complementary to each other at that position.

In general, the inhibitory nucleic acids useful in the methods described herein have at least 80% sequence complementarity to a target region within the target nucleic acid, e.g., 90%, 95%, or 100% sequence complementarity to the target region within a TBXT nucleic acid. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). An inhibitory nucleic acid (e.g., an ASO) that hybridizes to a TBXT target sequence can be identified through routine experimentation. In general, the inhibitory nucleic acids must retain specificity for their target, i.e., must not directly bind to, or directly significantly affect expression levels of transcripts other than the intended target.

### Antisense

As noted above, in some cases, an inhibitory nucleic acid of the present disclosure are ASOs. ASOs are typically designed to block expression of a DNA or RNA target by binding to the target and halting expression at the level of transcription, translation, or splicing. ASOs of the present disclosure are complementary nucleic acid sequences designed to hybridize under stringent conditions to a TBXT target sequence. Thus, oligonucleotides are chosen that are sufficiently complementary to the target, i.e., that hybridize sufficiently well and with sufficient specificity, to give the desired effect.

### Modified Bases/Locked Nucleic Acids (LNAs)

In some cases, an inhibitory nucleic acid of the present disclosure comprises one or more modified bonds or bases. Modified bases include phosphorothioate, methylphosphonate, peptide nucleic acids, or locked nucleic acid (LNA) molecules. For example, in some cases, the modified nucleotides are locked nucleic acid molecules, including [alpha]-L-LNAs. LNAs comprise ribonucleic acid analogues wherein the ribose ring is “locked” by a methylene bridge between the 2′-oxygen and the 4′-carbon—i.e., oligonucleotides, containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a TBXT target sequence. The LNA molecules can be chemically synthesized using methods known in the art.

### Antagomirs

In some cases, the inhibitory nucleic acid is an antagomir. Antagomirs are chemically modified antisense oligonucleotides that target a TBXT target nucleotide sequence. For example, an antagomir for use in the methods described herein can include a nucleotide sequence sufficiently complementary to hybridize to a TBXT target sequence of about 12 to 25 nucleotides, or from about 15 to 23 nucleotides.

In general, antagomirs include a cholesterol moiety, e.g., at the 3′-end. In some embodiments, antagomirs have various modifications for RNase protection and pharmacologic properties such as enhanced tissue and cellular uptake. For example, in addition to the modifications discussed above for antisense oligos, an antagomir can have one or more of complete or partial 2-O-methylation of sugar and/or a phosphorothioate backbone. Phosphorothioate modifications provide protection against RNase activity and their lipophilicity contributes to enhanced tissue uptake. In some embodiments, the antagomir can include six phosphorothioate backbone modifications; two phosphorothioates are located at the 5′-end and four at the 3′-end. Antagomirs useful in the present methods can also be modified with respect to their length or otherwise the number of nucleotides making up the antagomir. The antagomirs must retain specificity for their target, i.e., must not directly bind to, or directly significantly affect expression levels of, transcripts other than the intended target. In some embodiments, the inhibitory nucleic acid is locked and includes a cholesterol moiety (e.g., a locked antagomir).

siRNA/shRNA

In some cases, an inhibitory nucleic acid of the present disclosure is an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic-acid sequence or a portion thereof, and wherein the circular polynucleotide can be processed either in vivo or in vitro to generate an active siRNA molecule capable of mediating RNA interference.

In some cases, the interfering RNA coding region encodes a self-complementary RNA molecule having a sense region, an antisense region and a loop region. Such an RNA molecule when expressed desirably forms a “hairpin” structure, and is referred to herein as an “shRNA,” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology.

The target RNA cleavage reaction guided by siRNAs is highly sequence specific. In general, siRNA containing a nucleotide sequence identical to a portion of the target nucleic acid are used for inhibition. However, 100% sequence identity between the siRNA and the target gene is not required. Thus, the present disclosure has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general, the siRNAs must retain specificity for their target, i.e., must not directly bind to, or directly significantly affect expression levels of, transcripts other than the intended target.

### Making Inhibitory Nucleic Acids

Inhibitory nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

### Pharmaceutical Compositions

The present disclosure provides compositions, including pharmaceutical compositions, comprising an inhibitory nucleic acid of the present disclosure. An inhibitory nucleic acid of the present disclosure may be referred to below as an “agent” or an “active agent.”

In some cases, the compositions are formulated with a pharmaceutical acceptable carrier. The pharmaceutical compositions and formulations can be administered parenterally (e.g., intravenously or intramuscularly), topically, orally or by local administration, such as by intratumoral or peritumoral administration. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). An inhibitory nucleic acid can be formulated for administration, in any convenient way for use in human or veterinary medicine. Wetting agents, emulsifiers and lubricants, such as sodium lauryl sulfate and magnesium stearate, as well as coloring agents release agents, coating agents, sweetening, flavoring and perfuming agents, preservatives and antioxidants can also be present in the compositions.

Formulations of an inhibitory nucleic acid of the present disclosure include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount, of active ingredient (e.g., an inhibitory nucleic acid of the present disclosure) which can be combined with a carrier material to produce a single dosage form will vary depending upon the host being treated, the particular mode of administration, e.g.; intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixed with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as tale or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., an inhibitory nucleic acid of the present disclosure) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more Sweetening agents, such as sucrose, aspartame or saccharin. Formulations can be adjusted for osmolarity.

In some cases, oil-based pharmaceuticals are used for administration of an inhibitory nucleic acid. Oil-based suspensions can be formulated by suspending ah active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, an injectable oil-in-water emulsion comprises a paraffin oil, a sorbitan monooleate, an ethoxylated sorbitan monooleate and/or an ethoxylated sorbitan trioleate.

The pharmaceutical compositions can be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75: 107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some cases, the pharmaceutical compositions can be delivered transdermally, by a topical route, formulated as applicator sticks, solutions, suspensions, emulsions, gels, creams, ointments, pastes jellies, paints, powders, and aerosols.

In some cases, the pharmaceutical compositions can be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some cases, the pharmaceutical compositions can be parenterally administered, such as by intravenous (IV) administration or administration into a body cavity or lumen of an organ. These formulations can comprise a solution of active agent (e.g., an inhibitory nucleic acid of the present disclosure) in a pharmaceutically acceptable carrier. Acceptable vehicles and solvents that can be employed are water and Ringer's solution, an isotonic sodium chloride. In addition, sterile fixed, oils can be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For intravenous administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally acceptable diluent or solvent, such as a solution of 1,3-butanediol. The administration can be by bolus or continuous infusion (e.g., substantially uninterrupted introduction into a blood vessel for a specified period of time).

In some cases, the pharmaceutical composition can be lyophilized. Stable lyophilized compositions comprising an inhibitory nucleic acid can be made by lyophilizing a solution comprising a pharmaceutical composition of the present disclosure and a bulking agent, e.g., mannitol, trehalose, raffinose, and sucrose or mixtures thereof. A process for preparing a stable lyophilized formulation can include lyophilizing a solution of about 2.5 mg/mL nucleic acid, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent (e.g., an inhibitory nucleic acid of the present disclosure) into target cells w vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used herein, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the present disclosure can be administered for prophylactic and/or therapeutic treatments. In some cases, for therapeutic applications, compositions are administered to a subject in need thereof (e.g., an individual who is at risk of (e.g., at greater risk than the general population) or has a disorder described herein) in an amount sufficient to cure, alleviate or partially arrest the clinical manifestations of the disorder or its complications; this can be called a therapeutically effective amount.

The amount of pharmaceutical composition adequate to accomplish this is a therapeutically effective dose. The dosage schedule and amounts effective for this use. i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Remington: The Science and Practice of Pharmacy, 21st ed., 2005). The state of the art allows the clinician to determine the dosage regimen for each individual patient, active agent (e.g., inhibitory nucleic acid) and disease or condition treated. Guidelines provided for similar compositions used as pharmaceuticals can be used as guidance to determine the dosage regiment, i.e., dose schedule and dosage levels.

Single or multiple administrations of formulations can be given depending on for example: the dosage and frequency as required and tolerated by the patient, the degree and amount of therapeutic effect generated after each administration (e.g., effect on blood glucose levels), and the like. The formulations should provide a sufficient quantity of active agent (e.g., inhibitory nucleic acid) to effectively treat, prevent or ameliorate conditions, diseases or symptoms.

In alternative embodiments, pharmaceutical formulations for oral administration are in a daily amount of between about 1 g to about 100 mg nucleic acid per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

In some embodiments, the methods described herein can include co-administration with other drugs or pharmaceuticals, e.g., compositions for reducing blood glucose levels. For example, the inhibitory nucleic acids can be co-administered with drugs for treating or reducing risk of a disorder described herein.

### Methods of Treatment

The present disclosure provides methods of inhibiting the proliferation of a carcinoma, and methods of treating a carcinoma, in an individual. The methods comprise administering to an individual in need thereof (e.g., an individual having a carcinoma) an effective amount of an inhibitory nucleic acid of the present disclosure, or a pharmaceutical composition comprising an inhibitory nucleic acid of the present disclosure.

In some cases, an effective amount of an inhibitory nucleic acid of the present disclosure is an amount that, when administered in one or more doses to an individual in need thereof, reduces the tumor mass/tumor volume in the individual. In some cases, an “effective amount” of an inhibitory nucleic acid is an amount that, when administered in one or more doses to an individual in need thereof, increases survival time of the individual. For example, in some cases, an “effective amount” of an inhibitory nucleic acid is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the inhibitory nucleic acid.

In some cases, an “effective amount” of an inhibitory nucleic acid is an amount that, when administered in one or more doses to an individual in need thereof, either as a monotherapy or as part of a combination therapy, reduces the overall tumor burden in the individual, i.e., the amount of cancer in the body, or alternatively, causes the total tumor burden in the patient to remain relatively stable for a sufficient period of time for the patient to have a confirmed “stable disease” as determined by standard RECIST criteria. See, e.g., Aykan and Özatli (2020) World J. Clin. Oncol. 11:53.

In some cases, an effective amount of an inhibitory nucleic acid is an amount that, when administered in one or more doses to an individual in need thereof, either as a monotherapy or as part of a combination therapy, causes the tumor size to be reduced by a sufficient amount, and for a sufficient period of time, for the patient to have a confirmed “partial response” as determined by standard Response Evaluation Criteria in Solid Tumors (RECIST) criteria.

In some cases, an effective amount of an inhibitory nucleic acid is an amount that, when administered in one or more doses to an individual in need thereof (e.g., an individual having a tumor), either as a monotherapy or as part of a combination therapy, causes the tumor size to be reduced by a sufficient amount, and for a sufficient period of time, for the patient to have a confirmed “complete response” as determined by standard RECIST criteria.

Carcinomas that can treated by a method disclosed herein include, but are not limited to, chordoma, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma. In some cases, the carcinoma is a chordoma.

### Dosages

A suitable dosage of an inhibitory nucleic acid can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. An inhibitory nucleic acid of the present disclosure may be administered in amounts between 1 ng/kg body weight and 20 mg/kg body weight per dose, or higher, e.g. from 0.1 mg/kg body weight to 10 mg/kg body weight, e.g. from 1 mg/kg body weight to 5 mg/kg or from 5 mg/kg body weight to 10 mg/kg body weight; from 10-15 mg/kg, or higher, however, doses below or above this exemplary range are envisioned.

The frequency of administration of an inhibitory nucleic acid can vary depending on any of a variety of factors, but generally speaking will be administered once a week, once every two weeks, once every three weeks, once every four weeks, once per month, or less frequently than once per month, e.g., once every five weeks, once every six weeks, once every two months, once every three months, etc., but also can be administered more frequently than once per week, e.g., twice per week (biw), three times per week (tiw), four times per week, five times per week, six times per week, every other day (qod), or daily (qd). In some cases, the inhibitory nucleic acid is administered once every three weeks. Administration generally should be stopped upon disease progression or unacceptable toxicity.

The duration of administration of an inhibitory nucleic acid can vary, depending on any of a variety of factors, e.g., patient response, etc. For example, an inhibitory nucleic acid can be administered over a period of time ranging from one month to about two months, from about two months to about four months, from about four months to about six months, from about six months to about eight months, from about eight months to about 1 year, from about 1 year to about 2 years, or from about 2 years to about 4 years, or more. In some cases, the inhibitory nucleic acid will continue to be dosed for at least as long as the patient continues to receive a clinically determined benefit, which can be from at least many months to multiple years.

### Routes of Administration

Suitable routes of administration include oral, rectal, nasal, pulmonary, topical, subcutaneous, intramuscular, intraperitoneal, intravenous, intradermal, intrathecal, epidural, intracranial, intraspinal, intratumoral, and peritumoral. In some cases, the route of administration is intramuscular. In some cases, the route of administration is intravenous. In some cases, the route of administration is intracranial. In some cases, the route of administration is intraspinal. In some cases, the route of administration is intratumoral.

### Combination Therapy

The present disclosure contemplates the use of an inhibitory nucleic acid of the present disclosure in combination with one or more additional agents (e.g., one or more additional active therapeutic agents) or other prophylactic or therapeutic modalities. In such combination therapy, the various active agents frequently have different mechanisms of action. Such combination therapy may be especially advantageous by allowing a dose reduction of one or more of the agents, thereby reducing or eliminating the adverse effects associated with one or more of the agents; furthermore, such combination therapy may have a synergistic therapeutic or prophylactic effect on the underlying disease, disorder, or condition.

In some cases, a method of the present disclosure for treating cancer in an individual comprises: a) administering an inhibitory nucleic acid of the present disclosure; and b) administering at least one additional therapeutic agent or therapeutic treatment. Suitable additional therapeutic agents include, but are not limited to, a small molecule cancer chemotherapeutic agent, and an immune checkpoint inhibitor. Suitable additional therapeutic treatments include, e.g., radiation, surgery (e.g., surgical resection of a tumor), and the like.

As used herein, “combination” is meant to include therapies that can be administered separately, for example, formulated separately for separate administration (e.g., as may be provided in a kit), and therapies that can be administered together in a single formulation (i.e., a “co-formulation”).

In certain cases, an inhibitory nucleic acid of the present disclosure and the at least one additional agent are administered or applied sequentially, e.g., where one agent is administered prior to one or more other agents. In other cases, an inhibitory nucleic acid of the present disclosure and the at least one additional agent are administered simultaneously, e.g., where two or more agents are administered at or about the same time; the two or more agents may be present in two or more separate formulations or combined into a single formulation (i.e., a co-formulation). Regardless of whether the two or more agents are administered sequentially or simultaneously, they are considered to be administered in combination for purposes of the present disclosure.

Suitable additional therapeutic agents include, e.g., cancer chemotherapeutic agents, immune checkpoint inhibitors, immunotherapeutic agents, and the like.

### Subjects

Subjects suitable for treatment with a method of the present disclosure include individuals who have been diagnosed as having a carcinoma, individuals who have been treated for a carcinoma but who failed to respond to the treatment, and individuals who have been treated for a carcinoma and who initially responded but subsequently became refractory to the treatment and/or whose disease progressed while on the prior treatment.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. An inhibitory nucleic acid that comprises a nucleotide sequence that is complementary to a target nucleotide sequence in a TBXT transcript, wherein the inhibitory nucleic acid comprises one or more locked nucleic acids (LNA).

Aspect 2. The inhibitory nucleic acid of aspect 1, wherein the inhibitory nucleic acid has a length of from about 15 nucleotides to about 30 nucleotides.

Aspect 3. The inhibitory nucleic acid of aspect 1, wherein the inhibitory nucleic acid has a length of from about 15 nucleotides to about 20 nucleotides.

Aspect 4. The inhibitory nucleic acid of any one of aspects 1-3, wherein the nucleotide sequence that is complementary to a nucleotide sequence in a TBXT transcript is complementary to a nucleotide sequence in the intron 4/exon 5 junction.

Aspect 5. The inhibitory nucleic acid of any one of aspects 1-3, wherein the target TBXT nucleotide sequence is selected from:

(SEQ ID NO: 1)
CAGATCACAGCTCTTA;
(SEQ ID NO: 2)
AGATCACAGCTCTTAAA;
(SEQ ID NO: 3)
ATCACAGCTCTTAAAATT;
(SEQ ID NO: 4)
TTTTCAGATCACAGCTC;
(SEQ ID NO: 5)
TTTCAGATCACAGCTC;
(SEQ ID NO: 6)
TTTCAGATCACAGCTCT;
(SEQ ID NO: 7)
TTCAGATCACAGCTC;
and
(SEQ ID NO: 8)
TTCAGATCACAGCTCT.

Aspect 6. The inhibitory nucleic acid of any one of aspects 1-5, wherein the inhibitory nucleic acid comprises one or more phosphorothioate linkages.

Aspect 7. The inhibitory nucleic acid of any one of aspects 1-6, wherein the inhibitory nucleic acid comprises a nucleotide sequence selected from:

+T*+A*A*G*+A*G*C*+T*G*T*+G*A*T*+C*+T*+G;
+T*+T*T*A*+A*G*A*+G*C*T*+G*T*G*+A*T*+C*+T;
+A*+A*T*T*+T*T*A*+A*G*A*+G*C*T*+G*T*+G*+A*+T;
+G*+A*+G*C*+T*G*T*+G*A*T*+C*+T*G*+A*A*+A*+A;
+G*+A*G*+C*+T*+G*T*+G*A*T*+C*T*G*+A*+A*+A;
+G*+A*G*C*+T*G*T*+G*A*+T*+C*T*+G*+A*+A*+A;
+A*+G*+A*G*C*+T*G*T*+G*A*T*+C*T*G*+A*+A*+A;
+A*+G*+A*G*C*+T*G*T*+G*A*T*+C*T*+G*A*+A*+A;
+G*+A*G*C*+T*G*+T*+G*A*T*+C*T*G*+A*+A;
and
+A*+G*+A*G*C*+T*G*T*+G*A*T*+C*T*G*+A*+A,

wherein * indicates a phosphorothioate linkage, and wherein “+” immediately preceding a nucleotide indicates that the nucleotide is an LNA.

Aspect 8. A composition comprising:
a) an inhibitory nucleic acid of any one of aspects 1-7; and
b) a pharmaceutically acceptable excipient.

Aspect 9. The pharmaceutical composition of aspect 8, wherein the pharmaceutically acceptable excipient comprises one or more lipids.

Aspect 10. The pharmaceutical composition of aspect 8, wherein the pharmaceutically acceptable excipient comprises poly(amidoamine), poly(propyleneimine), or poly(L-lysine).

Aspect 11. A lipid nanoparticle comprising:
a) an inhibitory nucleic acid of any one of aspects 1-7; and
b) a pharmaceutically acceptable excipient.

Aspect 12. A method of treatment, the method comprising administering to an individual in need thereof an effective amount of an inhibitory nucleic acid of any one of aspects 1-7, a pharmaceutical composition of any one of aspects 8-10, or a lipid nanoparticle of aspect 11.

Aspect 13. A method of inhibiting proliferation of a carcinoma in an individual, the method comprising administering to the individual an effective amount of an inhibitory nucleic acid of any one of aspects 1-7, a pharmaceutical composition of any one of aspects 8-10, or a lipid nanoparticle of aspect 11.

Aspect 14. The method of aspect 13, wherein the carcinoma is a lung carcinoma, a breast carcinoma, a colon carcinoma, a prostate carcinoma, or a liver carcinoma.

Aspect 15. The method of aspect 13, wherein the carcinoma is a chordoma.

Aspect 16. A method of treating a carcinoma in an individual, the method comprising administering to the individual an effective amount of an inhibitory nucleic acid of any one of aspects 1-7, a pharmaceutical composition of any one of aspects 8-10, or a lipid nanoparticle of aspect 11.

Aspect 17. The method of aspect 16, wherein the carcinoma is a lung carcinoma, a breast carcinoma, a colon carcinoma, a prostate carcinoma, or a liver carcinoma.

Aspect 18. The method of aspect 16, wherein the carcinoma is a chordoma.

Aspect 19. The method of any one of aspects 16-18, wherein said administering comprises an intravenous, an intramuscular, an intratumoral, or a peritumoral route of administration.

Aspect 20. The method of any one of aspects 16-19, further comprising administering one or more additional therapeutic treatments.

Aspect 21. The method of aspect 20, wherein the one or more additional therapeutic treatments comprise cancer chemotherapy, radiation, or surgery.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or se, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Methods

### Cell Culture

Chordoma cell lines were obtained from ATCC, and maintained in media containing a 4:1 IMDM:RPMI ratio plus 10% FBS and non-essential amino acids and glutamine supplementation. All cell lines were grown on tissue culture plastic except for UCH-2, which is grown on Type I collagen-coated flasks. The U2OS cell line was obtained from the UC Berkeley Cell Culture Facility and maintained in DMEM high glucose+10% FBS.

### Antisense Oligonucleotides

ASO design was carried out by a contracted third party, and ordered from IdT Technologies. Lyophilized ASOs were resuspended in water at 200 μM and stored at −20 C. ASOs were diluted further in cell culture PBS for assays. A scrambled control mixmer LNA ASO (#406, sequence:

+C*G*+T*T*+A*G*+A*+T*T*+A*+C*/iMe-dC/*G*+C*+G).

### ASO Transfection

Cells were plated at 13,158 cells/cm2 and allowed to adhere for at least 24 hours or until 70% confluency was reached. Then ASOs were diluted in Opti-Mem and transfected via Lipofectamine 3000 per manufacturer's instructions. Final ASO concentration in the media ranged from 0.1 nM to 50 nM. Cells were incubated 48 to 96 hours, at which point media was removed, cells washed in PBS, and plates frozen at −80 C.

### Western Blot

Cell lysates were collected in 1× Laemmli buffer plus beta mercaptoethanol. Samples were separated by molecular weight via the Bio-Rad Mini Protean Tetra gel electrophoresis system. Protein was transferred to membranes using the Bio-Rad Trans-Blot Turbo Transfer System, then blocked in 5% milk for 1 hour. Membranes were incubated overnight in either a human brachyury primary antibody (R&D Systems, AF2085) or a Histone H3 primary antibody (Cell Signaling Technologies, 9715). Membranes were washed in TBS-Tween, then incubated 1 hour in anti-goat or anti-rabbit secondary antibodies, respectively. Following washing, membranes were treated with the SuperSignal West Pico Plus chemiluminescent substrate, then imaged on an iBright 1500 blot imager.

### Nonspecific Toxicity Assay

U2OS cells were plated at 7,813 cells/cm² in white opaque 96-well plates, and allowed to adhere for at least 24 hours or until 70% confluency was reached. ASOs were transfected at 50 nM in triplicate as previously described, and plates were incubated for 6 days, with media refreshed at 3 days. An equal volume of Cell Titer Glo 2.0 (Promega) was added to each well per manufacturer's instructions, and luminescence was measured on a Biotek Synergy LX platereader. Luminescence readings were normalized to an ASO-free or scrambled ASO control, and data were analyzed via one-way ANOVA followed by Dunnett's multiple comparison test.

### Chordoma Growth Inhibition Assay

Cells were plated and transfected as previously described, but in a large format 12-well plate. Following 72 incubation, each treatment was trypsinized, counted, and reseeded at 1,563 cells/cm2. Cells were allowed to grow for 21 days, with media refreshed every 3-4 days. At each time point, an equal volume of Cell Titer Glo 2.0 was added to each well and cell viability was assessed as described for nonspecific toxicity testing. Data were analyzed via one-way ANOVA followed by Dunnett's multiple comparison test.

### Results

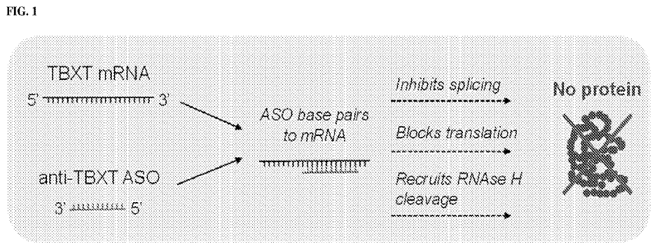

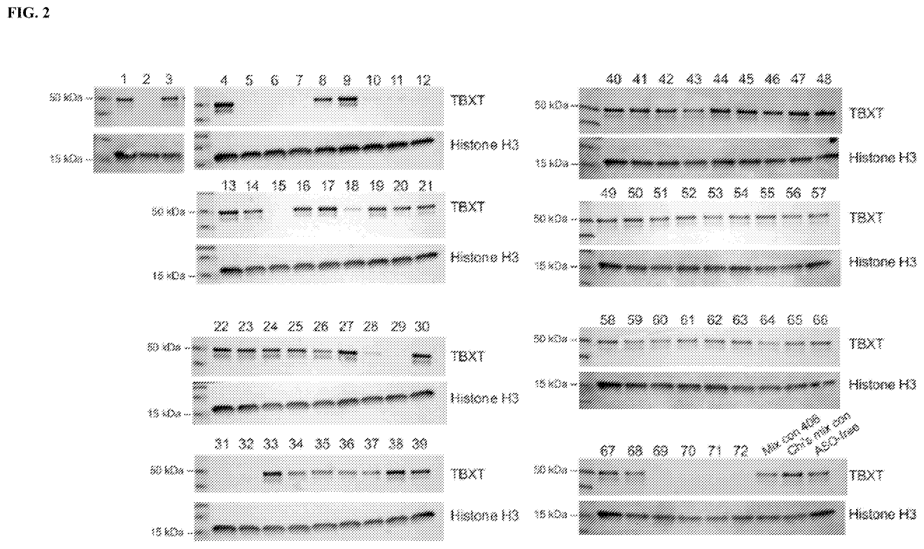

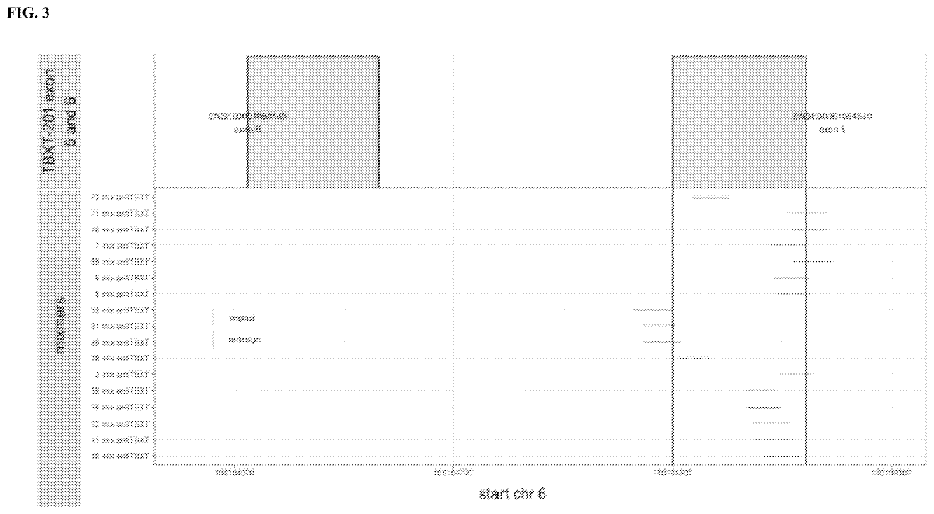

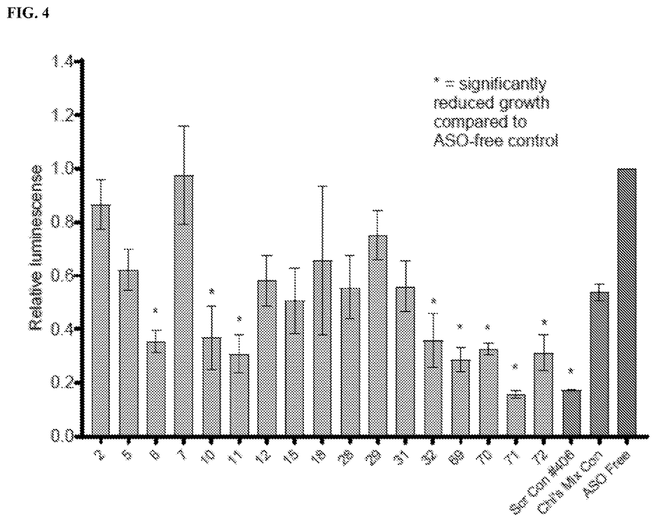

A library of 147 LNA ASOs (75 gapmers; 72 mixmers) was designed; this library may be referred to below as the “original” library. The LNA ASOs bind along the human brachyury (TBXT) pre-mRNA (NCBI Gen ID:6862). FIG. 1 schematically depicts the TBXT-targeting mechanism of the ASO library. Upon screening in the chordoma cell line UM-Chor1, 17 mixmer ASOs effectively abolished TBXT expression. FIG. 2. All 17 ASOs localized to the same exon of the TBXT pre-mRNA. FIG. 3. ASOs that exhibited nonspecific toxicity were eliminated using the TBXT-independent cell line U2OS, resulting in 9 remaining hits. FIG. 4. Dose-response testing determined that the most potent hits span the intron 4/exon 5 junction of the TBXT pre-mRNA, suggesting an exon skipping mechanism that is lethal to the protein. FIG. 5; FIG. 3.

FIG. 1. TBXT-targeting mechanism of the ASO library. ASOs are designed to base pair at various sites along the TBXT pre-mRNA. Gapmer ASOs induce RNAse H-mediated mRNA cleavage, while mixmer ASOs sterically inhibit splicing or translation. The end result is an absent or nonfunctional brachyury protein product.

FIG. 2. Select mixmer LNA ASOs effectively inhibit TBXT expression. UM-Chor1 cells were transfected with each ASO at 25 nM. After 48 h, cell lysates were probed for brachyury via Western blot. Absence of the 50 kDa protein indicates mixmer #2, 5, 6, 7, 10, 11, 12, 15, 18, 28, 29, 31, 32, 69, 70, 71, and 72 as hits. This experiment was repeated once with the same result.

FIG. 3. All anti-TBXT LNA ASO mixmer hits localize to the same exon. A comparison of target sites for the 17 ASOs capable of inhibiting TBXT protein reveals that all “hits” bind on and around exon 5 of the premRNA associated with TBXT transcript variant 1 (NCBI accession #: NM_003181).

FIG. 4. Non-specific toxicity of anti-TBXT LNA ASO mixmer hits. The TBXT-independent cell line U2OS was transfected with each ASO at 50 nM. At 6 days, Cell Titer Glo was added and luminescence was measured and normalized to an ASO-free control. Three replicates were measured and compared to the ASO-free control by one-way ANOVA and Dunnett's multiple comparison's test. A significant decrease in luminescence indicates a decrease in ATP and therefore decreased cell viability.

FIG. 5. Select mixmer LNA ASOs potently inhibit TBXT expression. UM-Chor1 cells were transfected with each ASO the indicated concentration. After 48 h, cell lysates were probed for brachyury via Western blot. Dose-response testing shows effective TBXT inhibition down to 10 nM in ASOs #2, 5, and 7.

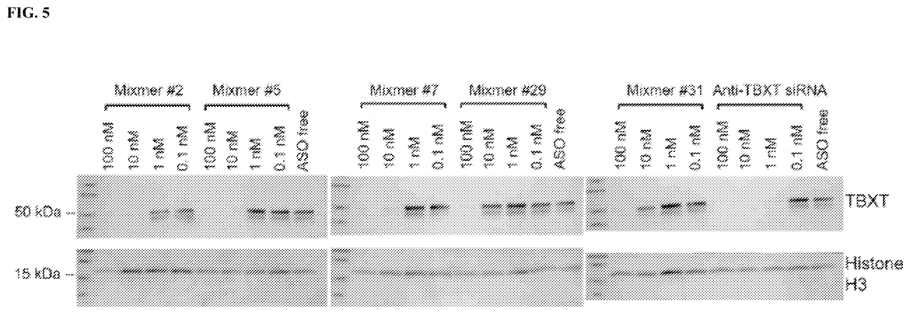

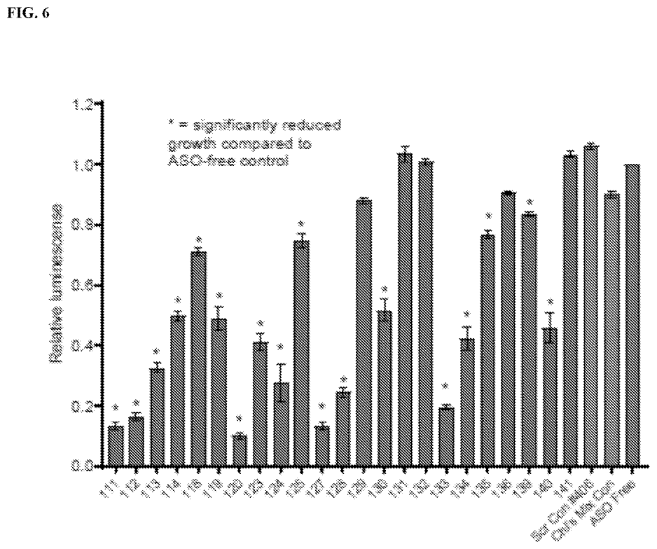

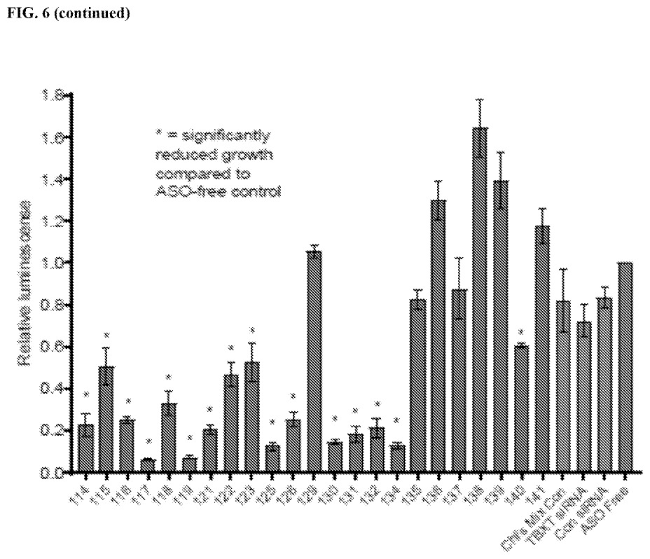

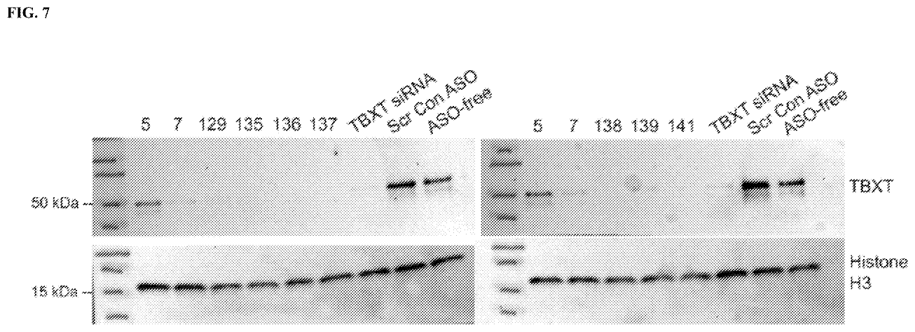

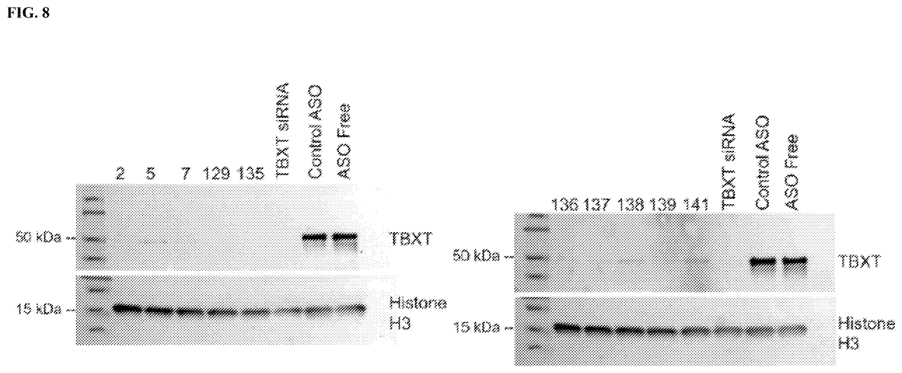

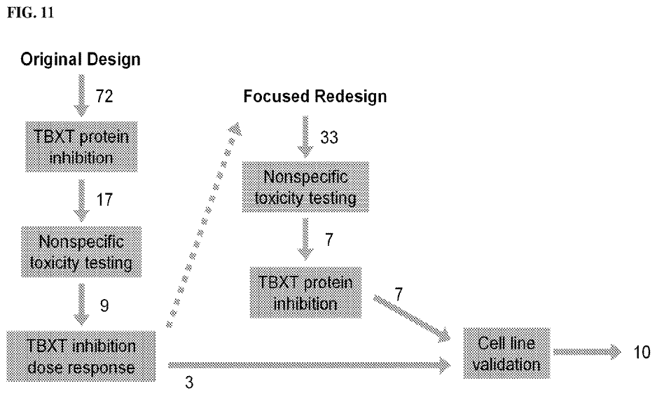

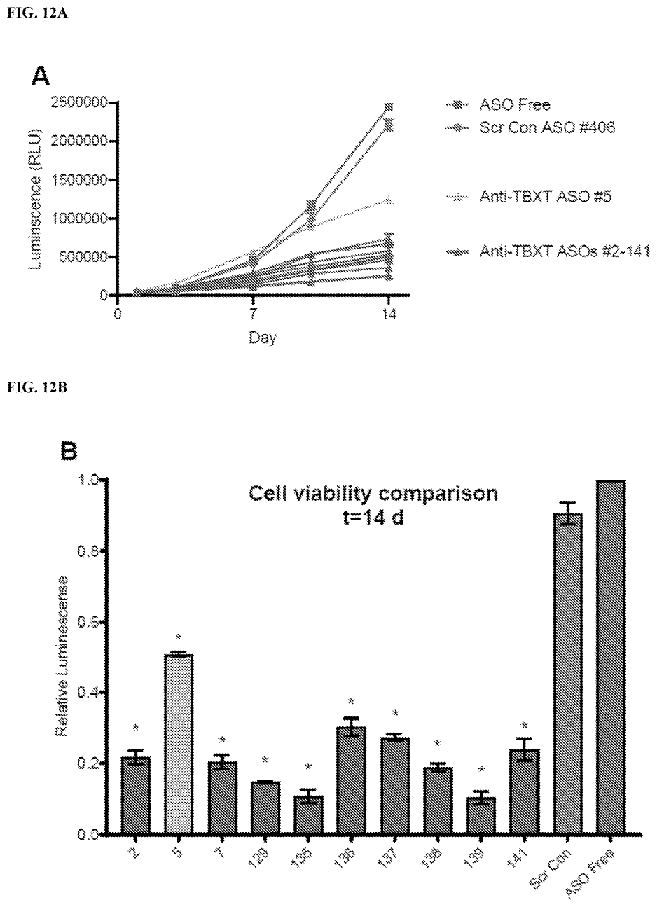

A second library of 33 LNA ASOs spanning the intron 4/exon 5 junction of TBXT was designed; this library may be referred to below as the “redesign” library. Non-specific toxicity testing identified 7 nontoxic hits (FIG. 6), which were confirmed to inhibit TBXT protein in the UM-Chor1 chordoma cells. FIG. 7. Dose response testing determined that the redesigns have equivalent TBXT inhibitory potency to ASOs #2, 5, and 7. The hits resulting from both the original design and the redesign were then validated in three additional chordoma cell lines, MUG-Chor1, JHC7, and UCH-2 and demonstrated broad ability to inhibit TBXT protein. FIG. 8-10. See FIG. 11 for a summary schematic of the ASO screen. The 10 anti-TBXT LNA ASO hits (3 original plus 7 redesign) were then tested for their ability to inhibit chordoma cell growth, as brachyury has been shown to be crucial for chordoma proliferation. All 10 ASOs were capable of significantly inhibiting the growth of UM-Chor1 cells (FIG. 12). See Table 1 for the sequences associated with the 10 anti-TBXT LNA ASO hits.

FIG. 6. Non-specific toxicity of anti-TBXT LNA ASO mixmer redesigns. The TBXT-independent cell line U2OS was transfected with each ASO at 50 nM. At 6 days, Cell Titer Glo was added, and luminescence was measured and normalized to an ASO-free control. Three replicates were measured and compared to the ASO-free control by one-way ANOVA and Dunnett's multiple comparison's test. A significant decrease in luminescence indicates a decrease in ATP and therefore decreased cell viability.

FIG. 7. All nontoxic mixmer LNA ASO redesigns effectively inhibit TBXT expression. UM-Chor1 cells were transfected with each ASO at 25 nM. After 48 h, cell lysates were probed for brachyury via Western blot. Absence of the 50 kDa protein indicates mixmer #129, 135, 136, 137, 138, 139, and 141 as hits.

FIG. 8. All original and redesign mixmer LNA ASO hits effectively inhibit TBXT expression in MUG-Chor1 cells. MUG-Chor1 cells were transfected with each ASO at 25 nM. After 96 h, cell lysates were probed for brachyury via Western blot. Absence of the 50 kDa protein indicates mixmer #2, 5, 7, 129, 135, 136, 137, 138, 139, and 141 as capable of inhibiting TBXT across multiple cell chordoma lines.

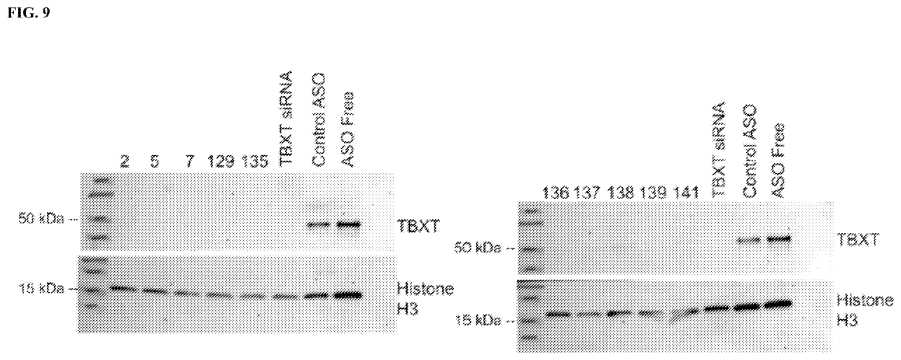

FIG. 9. All original and redesign mixmer LNA ASO hits effectively inhibit TBXT expression in JHC7 cells. JHC7 cells were transfected with each ASO at 25 nM. After 96 h, cell lysates were probed for brachyury via Western blot. Absence of the 50 kDa protein indicates mixmer #2, 5, 7, 129, 135, 136, 137, 138, 139, and 141 as capable of inhibiting TBXT across multiple cell chordoma lines.

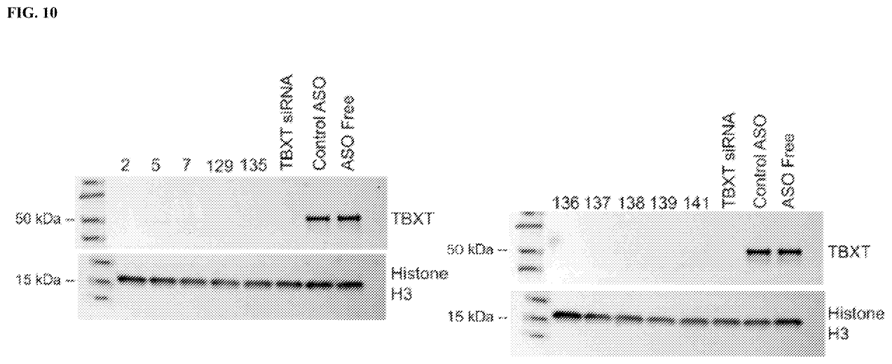

FIG. 10. All original and redesign mixmer LNA ASO hits effectively inhibit TBXT expression in UCH-2 cells. UCH-2 cells were transfected with each ASO at 25 nM. After 96 h, cell lysates were probed for brachyury via Western blot. Absence of the 50 kDa protein indicates mixmer #2, 5, 7, 129, 135, 136, 137, 138, 139, and 141 as capable of inhibiting TBXT across multiple cell chordoma lines.

FIG. 11. Anti-TBXT mixmer LNA ASO screen overview. 72 mixmer ASOs were tested for their ability to inhibit TBXT protein in the UM-Chor1 chordoma cell line. 17 hits emerged, 8 of which were eliminated for nonspecific toxicity. 9 ASOs were tested in dose response studies, and the 3 most potent ASOs enabled the identification of an optimal binding site on the TBXT pre-mRNA. An ASO redesign focused on this optimal site produced 33 new anti-TBXT mixmer LNA ASOs. 26 ASO redesigns for eliminated for nonspecific toxicity, with the remaining 7 displaying potent inhibition of TBXT. The 7 redesign hits combined with the 3 original design hits were validated in three independent chordoma cell lines, resulting in 10 overall hits.

FIG. 12A-12B. Anti-TBXT mixmer LNA ASOs inhibit chordoma cell growth. UM-Chor1 cells were Lipofectamine transfected with the anti-TBXT ASO hits. After 72 h, each treatment was trypsinized, counted, and reseeded in triplicate at a consistent number. (A) At the indicated time points, Cell Titer Glo was added, and luminescence was measured. (B) Luminescence values at t=14 days were normalized to an ASOfree control. Three replicates were measured and compared to the scrambled mixmer control by one-way ANOVA and Dunnett's multiple comparison's test. A significant decrease in luminescence indicates a decrease in ATP and therefore decreased cell viability.

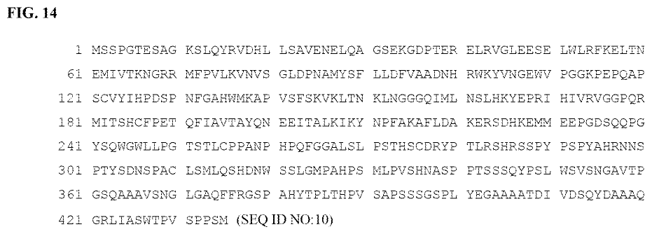

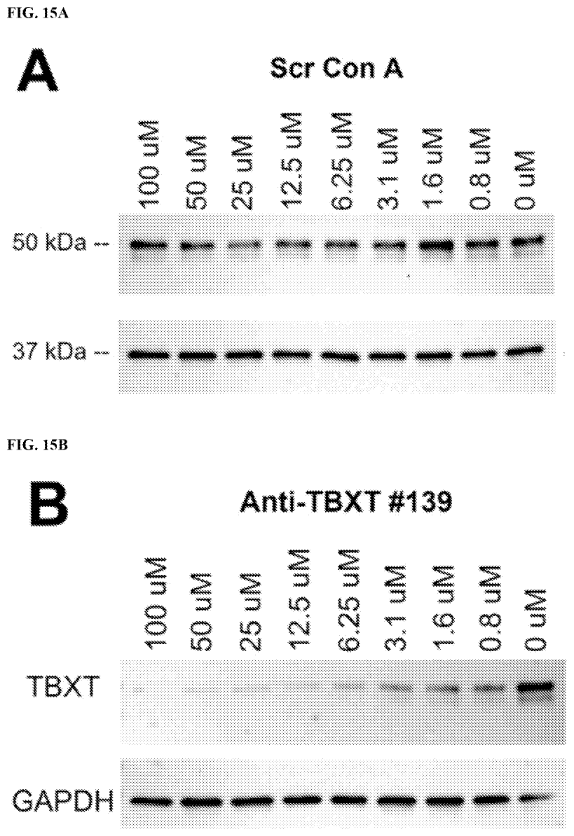

FIG. 15A-15B. Mixmer #139 enters chordoma cells without a lipid delivery vehicle to inhibit TBXT expression. UM-Chor1 cells were plated and ASO #139 (B) or scrambled control ASO (A) was added directly to the culture medium at the indicated concentration. Cells were incubated for 12 days, with ASO and media refreshed every 3-4 days. Cell lysates were then probed for brachyury via Western blot. Absence of the 50 kDa protein with increasing concentration of mixmer #139 indicates that the anti-TBXT ASO is capable of passively entering chordoma cells.

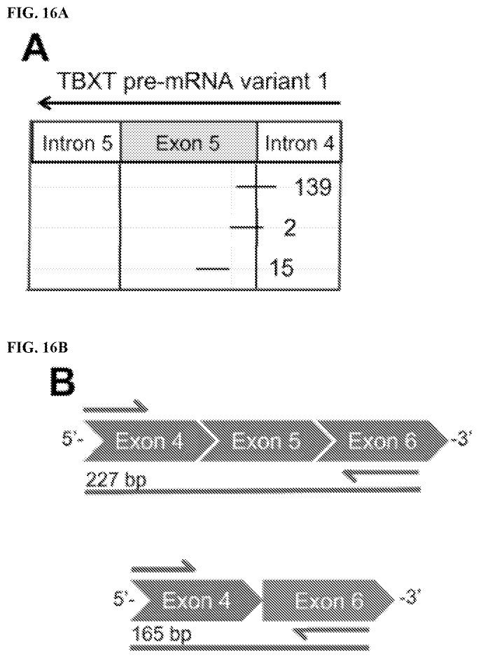

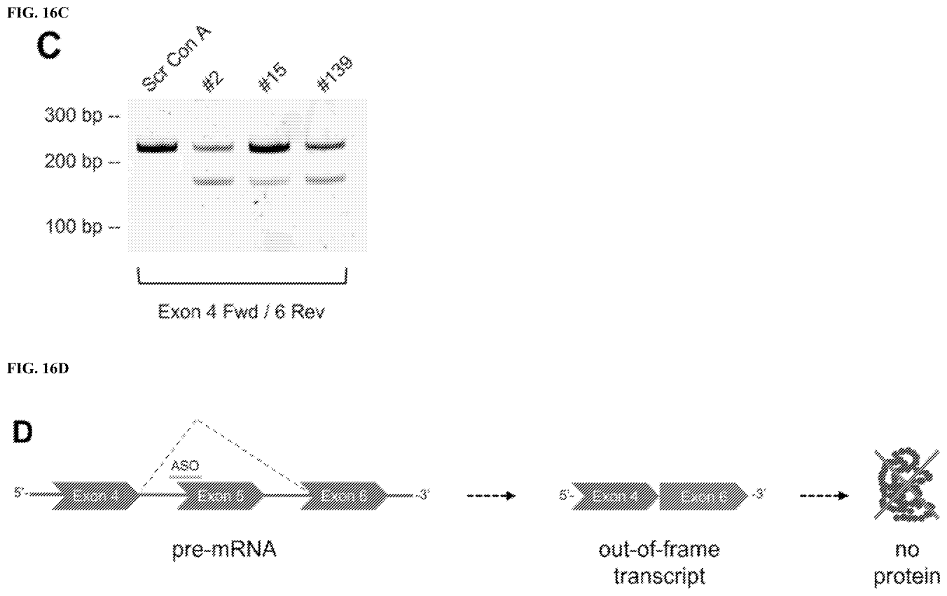

FIG. 16A-16D. ASOs directed to the intron 4/exon 5 junction of TBXT induce exon skipping and a lethal frame shift mutation. UM-Chor1 cells were Lipofectamine-transfected with mixmers #2, 15, 139, or scrambled control mixmer (A) at 25 nM and incubated for 72 h. RNA was extracted and the sequence encompassing the ASO binding site was PCR amplified using the indicated primers (B), then visualized on an acrylamide gel (C). A 165 base pair band in all anti-TBXT ASOs indicated loss of Exon 5. The primer products were excised and sequenced, and sequencing data confirmed precise skipping of TBXT Exon 5, resulting in a premature stop codon. A schematic (D) of the anti-TBXT ASO induced exon skipping and generation of a frameshift mutation is shown.

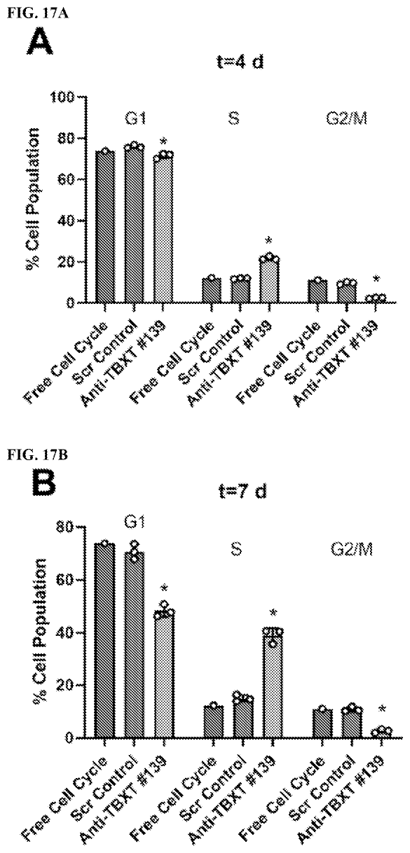

FIG. 17A-17B. Mixmer #139 induces an S-phase cell cycle arrest in chordoma cells. UM-Chor1 cells were Lipofectamine-transfected with anti-TBXT ASO #139 or scrambled control mixmer at 25 nM. Cells were incubated 48 h, then expanded and cultured for an additional 4 days (A) or 7 days (B) with media refreshed every 2-3 days. Cells were fixed and stained with propidium iodide, then analyzed via flow cytometry to estimate the percentages of cell populations in each phase of the cell cycle. A free cell cycle control from happily dividing cells was included as an additional control. Decreased cells in G1 with increased cells in S upon treatment with anti-TBXT #139 indicated an S-phase arrest, suggesting that TBXT suppression induces the replication stress checkpoint.

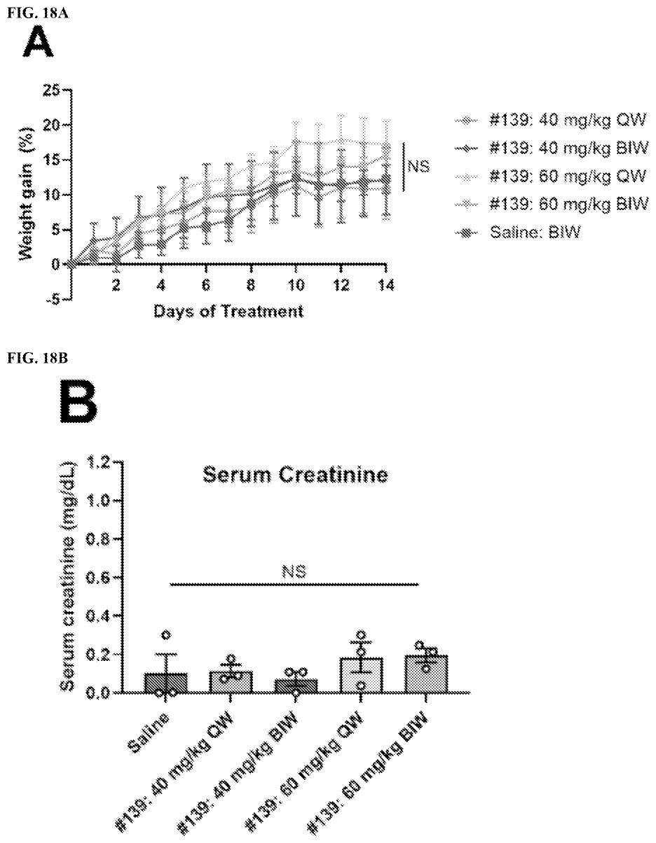

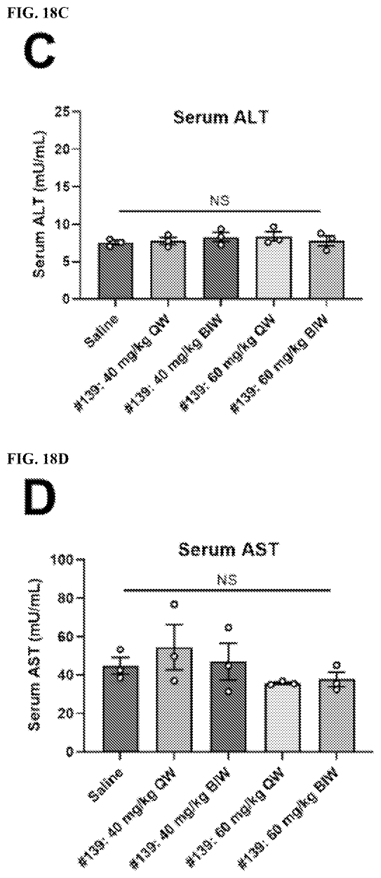

FIG. 18A-18E. Anti-TBXT ASO #139 is well tolerated in mice. Six week-old female nude mice were randomized by weight into treatment groups (3/group), and treated via s.c. injection with 40 or 60 mg/kg anti-TBXT ASO #139. A saline vehicle group was included as a negative control. Treatments were continued weekly or biweekly, as indicated, for 14 days, and body weights were measured daily and reported as a percent change from baseline (A). Serum was collected at study termination for measurement of creatinine (B), alanine aminotransferase (C), aspartate aminotransferase (D), and blood urea nitrogen (E). Treatment mouse body weights, as well as markers of liver/kidney toxicity, were not significantly different (NS) from the saline control, indicating high tolerability toward ASO #139.

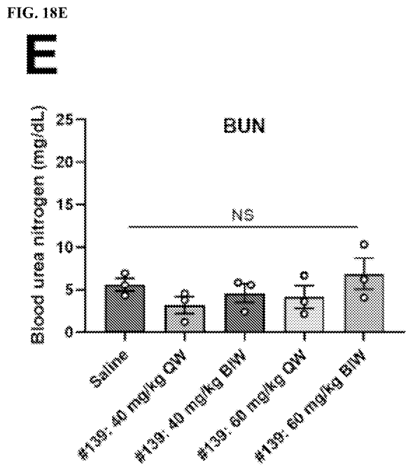

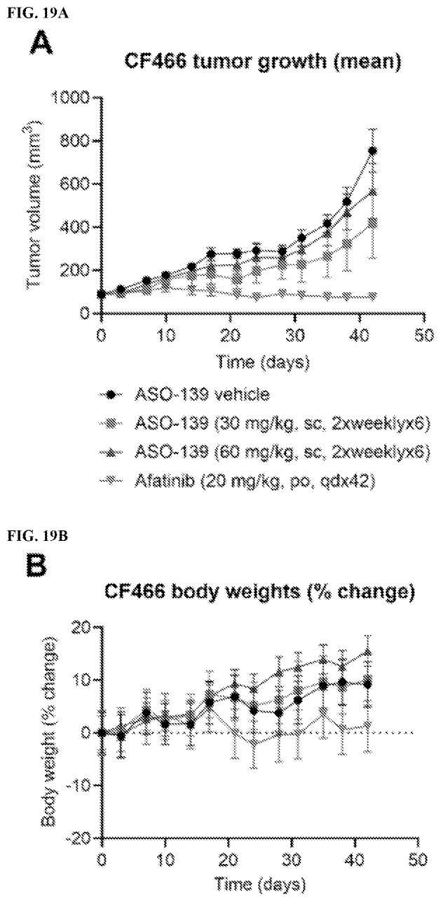

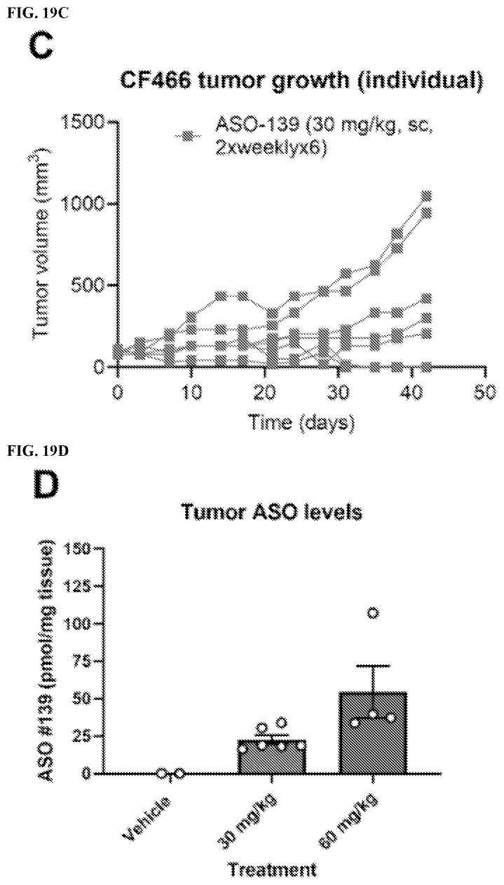

FIG. 19A-19D. Treatment with anti-TBXT ASO #139 selectively eradicates a patient-derived chordoma tumor in mice. Six week-old nude female mice were s.c. implanted with a 5×5 mm CF466 patient-derived chordoma tumor fragment. When tumors reached 100-200 mm³, mice were randomized into groups (7/group) and treatments were initiated. Anti-TBXT ASO #139 was s.c. injected biweekly at 30 or 60 mg/kg, with a saline vehicle group serving as a negative control. A positive control arm received 20 mg/kg afatinib. Body weights (B) and tumor dimensions (A, C) were measured twice per week, and treatments continued for six weeks. Body weights were reported as a % change from baseline. Tumors and livers were collected at study termination and flash frozen for quantification of ASO concentration. 10-20 mg samples of tumor were homogenized, and ASO #139 was quantified via SplintR qPCR using custom probes and primers for the target ASO. Mean cycle threshold values from an ASO standard curve were plotted against the log₁₀ of the concentration. A regression was fit to the linear portion of the curve, and ASO concentrations in tissue lysate samples were interpolated from these curves (D). 2/7 mice treated with 30 mg/kg ASO #139 experienced a complete tumor regression (C), indicating at least partial efficacy in this pilot study. ASO levels were detected in the remaining treatment mouse tumors, demonstrating robust accumulation of anti-TBXT ASO within a solid tumor upon systemic administration.

Table 1. Sequences associated with the anti-TBXT mixmer LNA ASO hits. Each ASO is a modified single-stranded DNA/LNA mixmer oligonucleotide. The phosphodiester backbone has been 100% substituted with a phosphorothioate backbone (indicated by “*” between each nucleotide under Sequence). The DNA monomers are intermixed with LNA monomers (a nucleotide preceded with a “+” designates an LNA). Target Sequence indicates the binding sequence, which spans the intron 4/exon 5 junction of the TBXT pre-mRNA transcript variant 1 (NCBI accession #: NM_003181).

TABLE 1

| | | Target |
|---|---|---|
| Name | Sequence | Sequence |
| 2 mix | +T*+A*A*G*+A*G*C* | CAGATCA |
| antiTBXT | +T*G*T*+G*A*T*+C* | CAGCTC |
| 5 mix | +T*+T*T*A*+A*G*A* | AGATCAC |
| antiTBXT | +G*C*T*+G*T*G*+A* | AGCTCT |
| 7 mix | +A*+A*T*T*+T*T*A* | ATCACAG |
| antiTBXT | +A*G*A*+G*C*T*+G*T* | CTCTTAA |
| 129 mix | +G*+A*+G*C*+T*G*T* | TTTTCAG |
| antiTBXT | +G*A*T*+C*+T*G*+A | ATCACA |
| 135 mix | +G*+A*G*+C*+T*+G*T* | TTTCAGA |
| antiTBXT | +G*A*T*+C*T*G*+A | TCACAG |
| 136 mix | +G*+A*G*C*+T*G*T*+ | TTTCAGA |
| antiTBXT | G*A*+T*+C*T*+G*+A | TCACAG |
| 137 mix | +A*+G*+A*G*C*+T*G* | TTTCAGA |
| antiTBXT | T*+G*A*T*+C*T*G*+ | TCACAG |
| 138 mix | +A*+G*+A*G*C*+T*G* | TTTCAGA |
| antiTBXT | T*+G*A*T*+C*T*+G* | TCACAG |
| 139 mix | +G*+A*G*C*+T*G*+T* | TTCAGAT |
| antiTBXT | +G*A*T*+C*T*G*+ | CACAG |
| 141 mix | +A*+G*+A*G*C*+T*G* | TTCAGAT |
| antiTBXT | T*+G*A*T*+C*T*G* | CACAGC |

Target Sequences in Table 1 are, from top to bottom, SEQ ID NOs:1-8, respectively.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An inhibitory nucleic acid that comprises a nucleotide sequence that is complementary to a target nucleotide sequence in a TBXT transcript, wherein the inhibitory nucleic acid comprises one or more locked nucleic acids (LNA).

2. The inhibitory nucleic acid of claim 1, wherein the inhibitory nucleic acid has a length of from about 15 nucleotides to about 30 nucleotides.

3. The inhibitory nucleic acid of claim 1, wherein the inhibitory nucleic acid has a length of from about 15 nucleotides to about 20 nucleotides.

4. The inhibitory nucleic acid of any one of claims 1-3, wherein the nucleotide sequence that is complementary to a nucleotide sequence in a TBXT transcript is complementary to a nucleotide sequence in the intron 4/exon 5 junction.

5. The inhibitory nucleic acid of any one of claims 1-3, wherein the target TBXT nucleotide sequence is selected from:
(SEQ ID NO: 1)
CAGATCACAGCTCTTA;
(SEQ ID NO: 2)
AGATCACAGCTCTTAAA;
(SEQ ID NO: 3)
ATCACAGCTCTTAAAATT;
(SEQ ID NO: 4)
TTTTCAGATCACAGCTC;
(SEQ ID NO: 5)
TTTCAGATCACAGCTC;
(SEQ ID NO: 6)
TTTCAGATCACAGCTCT;
(SEQ ID NO: 7)
TTCAGATCACAGCTC;
and
(SEQ ID NO: 8)
TTCAGATCACAGCTCT.

6. The inhibitory nucleic acid of any one of claims 1-5, wherein the inhibitory nucleic acid comprises one or more phosphorothioate linkages.

7. The inhibitory nucleic acid of any one of claims 1-6, wherein the inhibitory nucleic acid comprises a nucleotide sequence selected from:
+T*+A*A*G*+A*G*C*+T*G*T*+G*A*T*+C*+T*+G;
+T*+T*T*A*+A*G*A*+G*C*T*+G*T*G*+A*T*+C*+T;
+A*+A*T*T*+T*T*A*+A*G*A*+G*C*T*+G*T*+G*+A*+T;
+G*+A*+G*C*+T*G*T*+G*A*T*+C*+T*G*+A*A*+A*+A;
+G*+A*G*+C*+T*+G*T*+G*A*T*+C*T*G*+A*+A*+A;
+G*+A*G*C*+T*G*T*+G*A*+T*+C*T*+G*+A*+A*+A;
+A*+G*+A*G*C*+T*G*T*+G*A*T*+C*T*G*+A*+A*+A;
+A*+G*+A*G*C*+T*G*T*+G*A*T*+C*T*+G*A*+A*+A;
+G*+A*G*C*+T*G*+T*+G*A*T*+C*T*G*+A*+A;
and
+A*+G*+A*G*C*+T*G*T*+G*A*T*+C*T*G*+A*+A,
wherein * indicates a phosphorothioate linkage, and wherein “+” immediately preceding a nucleotide indicates that the nucleotide is an LNA.

8. A composition comprising:
a) an inhibitory nucleic acid of any one of claims 1-7; and
b) a pharmaceutically acceptable excipient.

9. The pharmaceutical composition of claim 8, wherein the pharmaceutically acceptable excipient comprises one or more lipids.

10. The pharmaceutical composition of claim 8, wherein the pharmaceutically acceptable excipient comprises poly(amidoamine), poly(propyleneimine), or poly(L-lysine).

11. A lipid nanoparticle comprising:
a) an inhibitory nucleic acid of any one of claims 1-7; and
b) a pharmaceutically acceptable excipient.

12. A method of treatment, the method comprising administering to an individual in need thereof an effective amount of an inhibitory nucleic acid of any one of claims 1-7, a pharmaceutical composition of any one of claims 8-10, or a lipid nanoparticle of claim 11.

13. A method of inhibiting proliferation of a carcinoma in an individual, the method comprising administering to the individual an effective amount of an inhibitory nucleic acid of any one of claims 1-7, a pharmaceutical composition of any one of claims 8-10, or a lipid nanoparticle of claim 11.

14. The method of claim 13, wherein the carcinoma is a lung carcinoma, a breast carcinoma, a colon carcinoma, a prostate carcinoma, or a liver carcinoma.

15. The method of claim 13, wherein the carcinoma is a chordoma.

16. A method of treating a carcinoma in an individual, the method comprising administering to the individual an effective amount of an inhibitory nucleic acid of any one of claims 1-7, a pharmaceutical composition of any one of claims 8-10, or a lipid nanoparticle of claim 11.

17. The method of claim 16, wherein the carcinoma is a lung carcinoma, a breast carcinoma, a colon carcinoma, a prostate carcinoma, or a liver carcinoma.

18. The method of claim 16, wherein the carcinoma is a chordoma.

19. The method of any one of claims 16-18, wherein said administering comprises an intravenous, an intramuscular, an intratumoral, or a peritumoral route of administration.

20. The method of any one of claims 16-19, further comprising administering one or more additional therapeutic treatments.

21. The method of claim 20, wherein the one or more additional therapeutic treatments comprise cancer chemotherapy, radiation, or surgery.
